(19) United States
(12) Patent Application Publication
(10) Pub. No.: US 2023/0078882 A1
(43) Pub. Date: Mar. 16, 2023

(54) DEVICE AND METHOD FOR ADJUSTING A QUANTITY OF ACTIVE SUBSTANCE INHALED BY A USER

(71) Applicant: ENOVAP, Rosieres Pres Troyes (FR)

(72) Inventors: MARIE HARANG-ELTZ, Rosieres Pres Troyes (FR); ALEXANDRE SCHECK, Rosieres Pres Troyes (FR); ALEXIS FISCHER, Rosieres Pres Troyes (FR); JULIEN ABULFEDA, Rosieres Pres Troyes (FR)

(21) Appl. No.: 17/759,514

(22) PCT Filed: Jan. 27, 2021
(86) PCT No.: PCT/EP2021/051878

§ 371 Date: Jul. 27, 2022

(30) Foreign Application Priority Data
Jan. 27, 2020 (FR) FR2000768

(51) Int. Cl. A24F 40/30; A24F 40/10; A24F 40/42; A24F 40/46; A24F 40/57; A24F 40/485

(52) U.S. Cl. CPC A24F 40/30; A24F 40/10; A24F 40/42; A24F 40/46; A24F 40/57; A24F 40/485; A61M 15/06

(57) ABSTRACT

Disclosed is a device (10) for adjusting the quantity of two liquids that are aerosolised in order to be inhaled simultaneously by a user, characterised in that said device comprises:
two tanks (105, 110), a first tank comprising a first liquid and a second tank comprising a second liquid having at least one different property, each liquid being configured to be aerosolised when this liquid undergoes a determined physical interaction;
an end piece (115) for inhalation, by the user, of the aerosolised liquid coming from each tank,
two aerosolisation means (120, 125) for aerosolising the first and second liquid respectively, each tank being associated with one aerosolisation means;
a single autonomous source of electrical power (130) for supplying electrical power to each aerosolisation means,
a means (135) for determining a ratio of liquids to be aerosolised for each liquid and
a switching means (140) for alternately supplying each aerosolisation means with electrical power from the single autonomous power source, as a function of the determined ratio, the switching means comprising two pulse-width modulators installed in series or a single pulse-width modulator between the autonomous electrical power source and each aerosolisation means.

### TECHNICAL FIELD OF THE INVENTION

The present invention relates to a device and a method for adjusting a quantity of active substance inhaled by a user. It applies, among others, to the field of inhalation, electronic cigarettes, smoking cessation, inhalation of THC or other cannabinoids, or mixture of e-liquids.

### STATE OF THE ART

There are currently three types of electronic cigarettes. The first generation of electronic cigarettes consisted of versions that are disposable when they no longer contain e-liquid.

The so-called second-generation electronic cigarettes have a push button, and pressing on the button allows the user to activate an aerosolisation means for aerosolising e-liquid. The only possible adjustment by the user is therefore the heating time of the e-liquid to obtain the aerosolised liquid.

The so-called third-generation electronic cigarettes make it possible to adjust, often by means of an adjustment wheel, the flow of air to which the aerosolised liquid is mixed to create a volume of aerosolised liquid more or less concentrated in liquid. These models sometimes make it possible to also increase or reduce the power delivered by the device to produce more or less aerosolised liquid.

There are currently electronic cigarettes that can be classified as fourth-generation, in which two tanks containing different e-liquids can be activated to mix the e-liquids of the tanks into the aerosolised liquid.

The international patent application WO 2019/122876 is known, which discloses a switching of the electrical power applied to two heating resistors, each associated with a tank, by means of two pulse-width modulators installed in parallel. Similarly, the American patent application US2019/357596, has a similar switching without specifying the way in which the switching is performed.

In this type of device, each pulse-width modulator has two functions:
ensuring that the power applied to the heating resistor is sufficient to vaporise the liquid contained in the tank to produce a volume of vapour corresponding to the volume defined by the user; and
keeping the ratio between the two liquids defined by the user.

The use of two pulse-width modulators in parallel has the disadvantage of requiring a very high frequency, for example between 100,000 Hertz and 200,000 Hertz to supply each heating resistor with sufficient power to heat the liquid.

The pulse-width modulators operating at such frequencies are very expensive. In addition, the installation in parallel of two pulse-width modulators consumes more energy due to the high frequency required.

These devices all have the drawback of needing frequent electrical charging, since they consume a large amount of energy, and/or high manufacturing costs.

### PRESENTATION OF THE INVENTION

The present invention aims to remedy all or part of these drawbacks.

To this end, according to a first aspect, the present invention relates to a device for adjusting the quantity of two liquids that are aerosolised in order to be inhaled simultaneously by a user, which comprises:
two tanks, a first tank comprising a first liquid and a second tank comprising a second liquid having at least one different property, each liquid being configured to be aerosolised when this liquid undergoes a determined physical interaction;
an end piece for inhalation, by the user, of the aerosolised liquid coming from each tank;
two aerosolisation means for aerosolising the first and second liquid respectively, each tank being associated with one aerosolisation means;
a single autonomous source of electrical power for supplying electrical power to each aerosolisation means;
a means for determining a ratio of liquids to be aerosolised for each liquid; and
a switching means for alternately supplying each aerosolisation means with electrical power from the single autonomous power source, as a function of the determined ratio, the switching means comprising two pulse-width modulators installed in series between the autonomous electrical power source and each aerosolisation means.

Thanks to these provisions, the power from the electrical supply is distributed in succession between each aerosolisation means for alternately supplying them. In particular, the thermal inertia phenomenon of thermal resistors, when these act as electrical aerosolisation means, is used to reduce the quantity of electrical energy needed to heat two liquids simultaneously. The efficiency of the device is therefore improved without the user detecting the alternating electrical power supply to each aerosolisation means.

From the power supplied to an aerosolisation means stems directly or indirectly, for example, the heating temperature of a thermal resistor or an oscillation frequency of a grid for a nebuliser.

The present invention makes it possible to successively define the total power supplied to the set of aerosolisation means, then the distribution of this power for each aerosolisation means and therefore have a single power management system. In this way, a single high-frequency pulse-width modulator is needed, and this reduces the costs because a high-frequency pulse-width modulator is five to ten times more expensive than a pulse-width modulator operating at lower frequencies of the order of 100 Hertz.

As the power used by the pulse-width modulators depends on their operating frequency, the present device is also more energy efficient. The sizing of the battery can therefore be reduced in relation to a device comprising two pulse-width modulators installed in parallel. Or, with an equivalent battery, the battery discharges less rapidly, which therefore increases the efficiency of the device and the life of the battery.

Lastly, the present invention makes possible space savings, a longer duration of use before recharging the device, and a reduction in the device's manufacturing costs.

In some embodiments, the upstream pulse-width modulator defines a duty cycle for supplying electrical energy to the set of aerosolisation means by alternating between two states, referred to as “on” and “off”.

In some embodiments, the downstream pulse-width modulator adjusts an alternating electrical duty cycle of each aerosolisation means as a function of the ratio determined, by alternating between two states referred to as “left” and “right”.

These embodiments make it possible to control, firstly, the total power supplied to the set of aerosolisation means and, secondly, the distribution of this power to each aerosolisation means.

In some embodiments, the device that is the subject of the present invention also comprises a means for calculating an inhalation time and a means for adjusting the switching as a function of the inhalation time calculated.

These embodiments make it possible to adjust the switching to maintain the ratio determined throughout the inhalation, whose duration cannot be considered to be usually constant or whose flow-rate can be variable. For example, a user can increase its flow-rate—aspiration more or less strong—and/or its aspiration time during the inhalation.

In some embodiments, the calculated inhalation time is learned from user data.

The advantage of these embodiments is to adjust the switching as a function of the user's habits.

In some embodiments, the inhalation time is calculated according to an inhalation in progress and the switching adjustment means adjusts the switching dynamically.

These embodiments make it possible to maintain precisely the ratio of liquid inhaled by the user as defined by adjusting the supply variables during the inhalation and throughout its duration.

In some embodiments, the device that is the subject of the present invention also comprises a means for choosing a ratio between a quantity of air or a quantity of aerosolised liquid to be inhaled, and in which the end piece for inhalation comprises an air inlet and a means for closing the air inlet as a function of the ratio chosen.

The advantage of these embodiments is to be able to define a quantity of active substance to be inhaled by a user.

To this end, according to a second aspect, the present invention relates to a device for adjusting the quantity of two aerosolised liquids to be inhaled simultaneously by a user, which comprises:
two tanks, a first tank comprising a first liquid and a second tank comprising a second liquid having at least one different property, each liquid being configured to be aerosolised when this liquid undergoes a determined physical interaction;
an end piece for inhalation, by the user, of the aerosolised liquid coming from each tank;
two aerosolisation means for aerosolising the first and second liquid respectively, each tank being associated with one aerosolisation means;
a single autonomous source of electrical power for supplying electrical power to each aerosolisation means;
a means for determining a ratio of liquids to be aerosolised for each liquid; and
a switching means for alternately supplying each aerosolisation means with electrical power from the single autonomous power source, as a function of the determined ratio, the switching means comprising a single pulse-width modulator between the autonomous electrical power source and each aerosolisation means.

Thanks to these provisions, the power from the electrical supply is distributed in succession between each aerosolisation means for alternately supplying them. In particular, the thermal inertia phenomenon of thermal resistors, when these act as electrical aerosolisation means, is used to reduce the quantity of electrical energy needed to heat two liquids simultaneously. The efficiency of the device is therefore improved without the user detecting the alternating electrical power supply to each aerosolisation means.

From the power supplied to an aerosolisation means stems directly or indirectly, for example, the heating temperature of a thermal resistor or an oscillation frequency of a grid for a nebuliser.

The present invention makes it possible to define the total power supplied to the set of aerosolisation means and, simultaneously, the ratio of activation of each aerosolisation means. In this way, a single high-frequency pulse-width modulator is needed in the entire switching means, and this reduces the costs because a high-frequency pulse-width modulator is five to ten times more expensive than a pulse-width modulator operating at lower frequencies.

As the power used by the pulse-width modulators depends on their operating frequency, the present device is also more energy efficient. The sizing of the battery can therefore be reduced in relation to a device comprising two pulse-width modulators installed in parallel. Or, with an equivalent battery, the battery discharges less rapidly, which therefore increases the efficiency of the device and the life of the battery.

The present invention also makes possible space savings and a reduction in the device's manufacturing costs.

Lastly, such a device makes it possible to have greater accuracy in the evaporation of liquids from the two tanks, because the final power is obtained before being applied to one or other of the aerosolisation means. The switching period of the power is set and therefore improves the stability of the system.

With respect to the first aspect described above, fewer components are necessary thereby reducing the cost and increasing the compactness of the device, however the quantity of calculations for determining the various states is increased.

In some embodiments the pulse-width modulator has three supply states, “off”, “left” and “right”, and the pulse-width modulator adjusts:
a duty cycle for supplying electrical energy by alternating between the sum of the duration in the left and right states, and the duration in the off state; then
an alternating duty cycle for supplying each aerosolisation means with electrical power as a function of the ratio determined, by alternating between the two supply states, left and right.

These embodiments make it possible to control the total power supplied to the set of aerosolisation means and the distribution of this power to each aerosolisation means with a single pulse-width modulator.

In some embodiments, the switching means comprises a means for defining a switching period in which each aerosolisation means is supplied in succession.

Thanks to these provisions, the device can be adapted to any type of aerosolisation means and any type of liquid without the user detecting the switching during inhalation.

In some embodiments, the device that is the subject of the present invention also comprises a means for calculating an inhalation time and a means for adjusting the switching as a function of the inhalation time calculated.

These embodiments make it possible to adjust the switching to maintain the ratio determined throughout the inhalation, whose duration cannot be considered to be usually constant or whose flow-rate can be variable. For example, a user can increase its flow-rate—aspiration more or less strong—and/or its aspiration time during the inhalation.

In some embodiments, the calculated inhalation time is learned from user data.

The advantage of these embodiments is to adjust the switching as a function of the user's habits.

In some embodiments, the inhalation time is calculated according to an inhalation in progress and the switching adjustment means adjusts the switching dynamically.

These embodiments make it possible to maintain precisely the ratio of liquid inhaled by the user as defined by adjusting the supply variables during the inhalation and throughout its duration.

In some embodiments, the device that is the subject of the present invention also comprises a means for choosing a ratio between a quantity of air or a quantity of aerosolised liquid to be inhaled, and in which the end piece for inhalation comprises an air inlet and a means for closing the air inlet as a function of the ratio chosen.

The advantage of these embodiments is to be able to define a quantity of active substance to be inhaled by a user and to obtain different draught sensations, for example a draught referred to as “tight” or “aerial” known to the person skilled in the art.

According to a third aspect, the present invention relates to a method of adjusting the quantity of two aerosolised liquids to be inhaled simultaneously by a user, each liquid being contained in a tank associated with one aerosolisation means, which method comprises:
a step of determining a ratio of liquids to be aerosolised for each liquid;
a switching step for alternately supplying each aerosolisation means with electrical power from the single autonomous power source, by pulse-width modulation between the autonomous electrical power source and each aerosolisation means by the use of one pulse-width modulator or two pulse-width modulators installed in series;
a step of evaporating the liquid contained in each tank when this liquid undergoes a determined physical interaction; and
a step of the user inhaling aerosolised liquid coming from each tank.

As the particular aims, advantages and features of the method that is the subject of the present invention are similar to those of the devices that are the subjects of the present invention, they are not repeated here.

### BRIEF DESCRIPTION OF THE FIGURES

Other advantages, aims and particular features of the invention will become apparent from the non-limiting description that follows of at least one particular embodiment of the devices and the method that are the subjects of the present invention, with reference to drawings included in an appendix, wherein:

FIG. 1 represents, schematically, a first particular embodiment of the device that is the subject of the present invention wherein the switching means comprises two pulse-width modulators installed in series;

FIG. 2 represents, schematically, two heating curves of a thermal resistor acting as aerosolisation means;

FIG. 3 represents, schematically, a first embodiment of an electrical power supply to each aerosolisation means of the device that is the subject of the present invention;

FIG. 4 represents, schematically, a second embodiment of an electrical power supply to each aerosolisation means of the device that is the subject of the present invention;

FIG. 5 represents, schematically and in the form of a logic diagram, a particular series of steps of the method that is the subject of the present invention; and

FIG. 6 represents, schematically, a second particular embodiment of the device that is the subject of the present invention wherein the switching means comprises a single pulse-width modulator.

### DETAILED DESCRIPTION

The present description is given in a non-limiting way, in which each characteristic of an embodiment can be combined with any other characteristic of any other embodiment in an advantageous way.

Note that the figures are not to scale.

Note that the term “property” denotes, for example, a concentration of an active substance in a liquid, or a thermodynamic or chemical property of a liquid.

Note that the term “aerosolise” denotes any action consisting of suspending a liquid by vaporisation or nebulisation, for example.

FIG. 2 shows two heating curves 23 and 24 of a thermal resistor acting as aerosolisation means. These curves show the temperature of a thermal resistor acting as aerosolisation means 21 as a function of the heating duration. The inventors have noted that when a thermal resistor is supplied alternatively, curve 24, the heating duration needed to reach a predefined temperature 25 is very similar to the heating duration when a thermal resistor is supplied continuously with electrical current, given the thermal inertia of the thermal resistor.

In the case of nebulisation it is the frequency, not the power, that is managed by the pulse-width modulator sent on each nebuliser. The installations in parallel or in series of pulse-width modulators therefore make it possible to manage the frequency of each nebuliser in order to dose the quantity of aerosol it produces.

In the present invention, the inventors take of advantage of and apply this discovery by supplying two pulse-width modulators alternatively by switching between the supplying of one or other of the aerosolisation means. The energy consumption, the place required to supply the set of aerosolisation means, and the cost of the device are therefore reduced without affecting the volume of aerosolised liquid inhaled by the user.

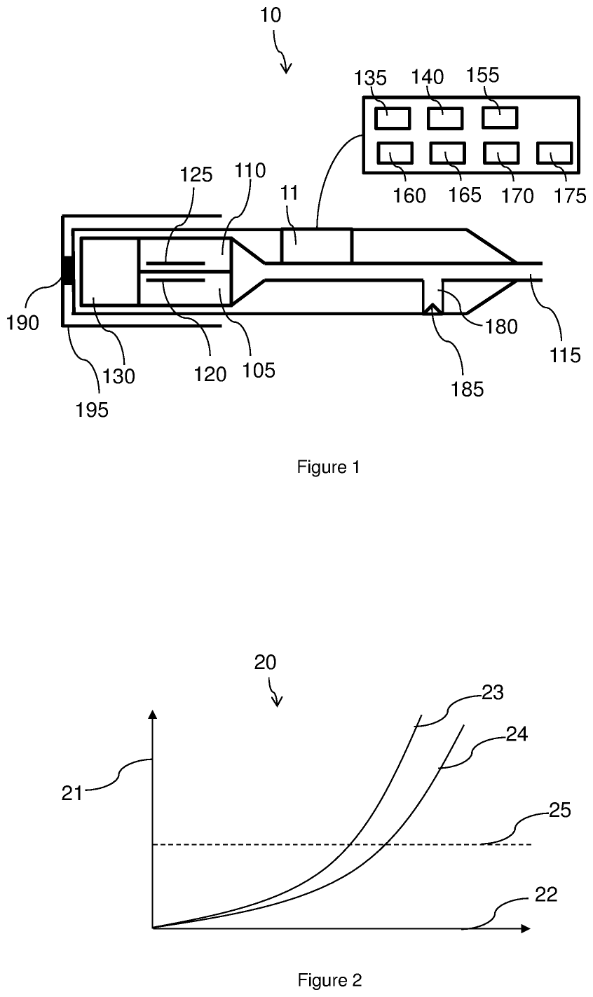

FIG. 1, which is not to scale, shows a schematic view of an embodiment of the device 10 that is the subject of the present invention.

FIG. 1, which is not to scale, shows a cross-section view of an embodiment of the device 10 that is the subject of the present invention. This device 10 comprises:
two tanks, 105 and 110, a first tank comprising a first liquid and a second tank comprising a second liquid having at least one different property, each liquid being configured to be aerosolised when this liquid undergoes a determined physical interaction;
an end piece 115 for inhalation, by the user, of the aerosolised liquid coming from each tank;
two aerosolisation means, 120 and 125, for aerosolising the first and second liquid respectively, each tank being associated with one aerosolisation means;
a single autonomous source of electrical power 130 for supplying electrical power to each aerosolisation means;
a means 135 for determining a ratio of liquids to be aerosolised for each liquid; and
a switching means 140 for alternately supplying each aerosolisation means with electrical power from the single autonomous power source, as a function of the determined ratio, the switching means comprising two pulse-width modulators, 155 and 160, installed in series between the autonomous electrical power source and each aerosolisation means.

The two tanks, 105 and 110, are, for example, two tanks with identical dimensions configured to be transportable in a device with dimensions comparable to those of an electronic cigarette. Preferably, each of these tanks, 105 and 110, comprises an incorporated aerosolisation means, 120 and 125. In some alternative embodiments, each tank comprises a cavity, not shown, making it possible to insert an aerosolisation means, 120 and 125. An aerosolisation means, 120 or 125, is associated with each tank, 105 or 110, such that when an aerosolisation means, 120 or 125, is activated, only the liquid contained in the associated tank, 105 or 110, is aerosolised.

Preferably, each tank, 105 and 110, comprises a removable cap for refilling with liquid to be aerosolised. The liquid contained in each tank can vary or be replaced during refill actions.

The liquids contained in each tank have at least one different characteristic or a combination of different characteristics. The characteristics of the liquid are, for example:
a taste;
an active substance, for example nicotine, Tetrahydrocannabinol (acronym “THC”), cannabidiol (acronym “CBD”), or a compound for therapeutic purposes;
a concentration of an active substance;
a viscosity;
a heating temperature for vaporising the liquid;
or any other known characteristic defining a liquid for an electronic cigarette.

In some variants, the two tanks, 105 and 110, are positioned parallel to each other along a general longitudinal axis of the device 100. This device comprises the inhalation end piece 115, downstream from a side of the air channel passing via an outlet of each tank, and an air inlet, not shown, upstream from the air channel.

In some variants, the device 10 comprises at least three tanks.

The inhalation end piece 115 is, for example, a duct configured to allow a user to inhale the aerosolised liquids exiting from the tanks, 105 and 110.

The two aerosolisation means, 120 and 125, are, for example, electrical resistors heating by Joule effect when a current is applied to the terminals of these aerosolisation means. The heating of such a resistor, 120 and 125, depends upon the amperage of the current passing through said aerosolisation mean, 120 and 125. Therefore, the heating of the resistor, 120 and 125, can be regulated by a control means 11 configured to apply current to each aerosolisation means, 120 and 125.

In some variants, each aerosolisation means, 120 and 125, can be a grid nebuliser whose agitation in the liquid at a higher or lower frequency results in the nebulisation of the liquid.

In some variants, each aerosolisation means, 120 and 125, can be a separate type.

The liquids contained in the tanks, 105 and 110, can have different predefined aerosolisation limit values. For example, according to the properties of the liquid, the minimum temperature required to vaporise the liquid can be different.

The single autonomous electrical power source 130 is preferably a rechargeable battery. The rechargeable batteries known to the person skilled in the art. In other embodiments, the single autonomous electrical power source 130 is an electrical cell or a set of electrical cells arranged in a way known to the person skilled in the art.

The autonomous electrical power source 130 supplies the two aerosolisation means indirectly and alternately. This means that a single source supplies the electrical amperage necessary for heating the aerosolisation means 120 and 125. In other words, the electrical aerosolisation means are both connected to the same autonomous electrical power source 130. The electrical current coming from the single autonomous power source and supplied to each aerosolisation means 120 and 125 is controlled by the control means 11. This means that the control means 11 distributes the electrical power to each aerosolisation means 120 and 125 according to elements defined below.

In some embodiments, the device 10 comprises a removable cover 195 for protecting the tanks, 105 and 110, this cover 195 comprising a means 190 for charging the single autonomous power source 130. This charging means 190 is, for example, an electrically conductive shank, for example a mini or micro USB (“Universal Serial Bus”), put into contact with a power supply shank (not shown) of the device 10. In some variants, this charging means 190 utilises induction charging. This cover 195 comprises, for example, an electrical power supply, such as, for example, a cell or a battery.

The control means 11 comprises the means 135 for determining a ratio of liquids to be aerosolised for each liquid. The determination means 135 is, for example, a computer program incorporated in a communicating portable terminal and/or in the device comprising the tanks, 105 and 110. In FIG. 1, the determination means is incorporated into the device 10. The communicating portable terminal is, for example, a smartphone or a digital tablet.

Preferably, the means of the control means are computer programs utilised by a microprocessor, in the device 10 or remote, for example in a smartphone.

In some embodiments, the determination means 135 comprises a means for setting, by a user, the ratio of liquids to be aerosolised. The adjustment means can comprise a display means, for example a screen, which indirectly controls a ratio of liquids to be aerosolised, the ratio of activation of the set of aerosolisation means not necessarily being proportional to said ratio of liquids to be aerosolised, and a means for controlling the ratio of liquids displayed and therefore adjusted. The adjustment means can be an adjustment wheel, or push or touch buttons bearing the inscriptions “+”, to increase the percentage of aerosolised liquid from the first tank 105, and “−”, to reduce said percentage.

In some embodiments, the determination means 135 comprises a means for accessing a user profile. This user profile corresponds to a standard user profile determined as a function of the user's consumption data collected by declaration or automatic training. These consumption data comprise, for example:
a consumption frequency as a function of a time of day or week;
a typical time of a day of consumption; and
distribution of the inhalation of aerosolised liquid coming from each liquid of the device 10.

Where the consumption data are learned, these data are obtained by accumulating memorised data relating to use of the control means. During a learning period, the control means 11 is, for example, configured to control the aerosolisation of a constant ratio of aerosolised liquid at constant regulation. Each inhalation is dated by a timestamping means, such as an electronic clock. Data representative of each inhalation are transmitted to a memory by means of a transmission means. This transmission means is, for example, an antenna configured to emit a wireless signal using Bluetooth technology (registered trademark), WiFi (registered trademark) or any other wireless technology known to the person skilled in the art. In some variants, the memory is in the same housing as the inhalation end piece. In other variants, the memory is incorporated into the communicating portable terminal. In other variants, the memory is remote.

Based upon memorised data, a means for determining a user profile determines a user profile. This means for determining a user profile is, for example, a computer program configured to compare a graph of consumption of each tank over time, on the scale of a day and/or a week, to standard consumption graphs, and records the settings of the proportion of aerosolised liquid produced by each tank. When a standard consumption graph that is the closest to the learned consumption graph is determined, the means for determining a user profile determines that the user profile associated to this standard graph corresponds to the standard profile of the user whose mode of consumption has been learned.

The access means is, for example, an antenna configured to communicate with a remote server holding data related to the user profile.

In some embodiments, the device 10 comprises a means 175 for choosing a ratio between a quantity of air or a quantity of aerosolised liquid to be inhaled, and in which the end piece for inhalation 115 comprises an air inlet 180 and a means 185 for closing the air inlet as a function of the ratio chosen.

The air inlet 180 is, for example, a through-hole through to the inhalation means. The closing means is, for example, a valve for opening or closing the through-hole of the air inlet 180 controlled electronically, or a rotating ring to make the through-hole of the air inlet 180 with an opening or a closed surface.

The means 175 for choosing can be chosen or learned automatically in the same way as the description above concerning the determination means 135. The means for determining 135 and choosing 175 can be a single means for controlling the ratio between liquids to be aerosolised, firstly, and the ratio between the sum of the quantities of aerosolised liquid and a quantity of air, secondly.

It can therefore be seen that a concentration of an element of each aerosolised liquid, for example a taste agent or an active substance, can be predefined in a volume of aerosolised liquid inhaled by the user.

In some embodiments, the determination means 135 is configured to determine a quantity of an active substance to be aerosolised as a function of a standard weaning graph associated to the standard user profile determined. This graph generally decreases over time on a scale of a week, for example. However, this graph can increase at certain times in a day or a week based upon the user's noticed habits of consumption.

An item of timestamp data is linked to a determination time by the determination means 135. This item of timestamp data is obtained, for example, by an electronic clock configured to measure an activation date and time of one of the means of the device 10.

The means 135 for determining a quantity of active substance to be aerosolised determines the quantity as a function of the user profile data.

The determination means 135 determines a quantity of active substance to be aerosolised as a function of an item of timestamp data related to a starting up of the device 10.

The determination means 135 determines an increasing quantity or concentration of active substance, relative to the last quantity of active substance determined, when the item of timestamp data is the first item of timestamp data greater than a predefined time. For example, the first inhalation of the day has a greater quantity of active substance than the last inhalation of the previous day.

In some variants, the determination means 135 determines an increasing quantity or concentration of active substance when a length of time longer than a predefined limit time has elapsed since the last inhalation.

The determination means 135 determines a generally-decreasing quantity of active substance as a function of the item of timestamp data.

The means for detecting the user's frequency of inhaling on the inhalation means 115 is, for example, an electronic circuit comprising a counter of the number of the inhalations completed by the user on the end piece for inhalation 115. The number of inhalations is determined, for example, by using a propeller configured to turn when the air passes through the duct of the inhalation means 115 in a predefined direction. This number of inhalations, measured over a rolling predefined limit time, divided by the rolling predefined limit time, gives an inhalation frequency.

When this inhalation frequency is greater than a predefined limit frequency, the determination means 130 determines an increasing quantity or concentration of active substance to be aerosolised, relative to the previous quantity of active substance determined. Generally, the determination means 130 determines the quantity or concentration of active substance as a function of the inhalation frequency detected.

The device 10 comprises a means for capturing the user's blood-alcohol level. This capture means is, for example, an alcohol sensor connected to the inhalation means 115.

The determination means 135 determines the quantity of active substance to be aerosolised as a function of the blood-alcohol level captured. If the blood-alcohol level captured is high and an item of data, variable or not, of the user profile indicates that the user is a driver, the determined quantity of active substance is increased. Conversely, if the user profile indicates that the user is not a driver, the determined amount of active substance is reduced.

In some variants, the determination means 135 is incorporated into the same housing as the end piece for inhalation 115. In other variants, the determination means 135 is in a remote memory, such as a server for example.

The determined quantity of active substance to be aerosolised is sent, by a means for emitting an item of information representative of the determined quantity or concentration of active substance, towards the switching means 140 and the possible closing means 185. This transmission means is, for example, an antenna of the communicating portable terminal comprising the determination means 135 configured to emit a wireless signal using Bluetooth technology (registered trademark), WiFi (registered trademark) or any other wireless technology known to the person skilled in the art.

The device 10 comprises a switching means 140 for alternately supplying each aerosolisation means with electrical power from the single autonomous power source and electronic components that manage the single power 130, as a function of the determined ratio.

The switching means 140 manages the electrical power supply to each aerosolisation means. The switching means 140 switches the electrical power supply between at least two states, one configured to supply electrical power to a first aerosolisation means 120, the other configured to supply electrical power to a second aerosolisation means 125. In some embodiments, the switching means 140 switches to a third state in which no aerosolisation means is supplied with electrical power.

The switching means 140 comprises two pulse-width modulators, 155 and 160, installed in series between the autonomous electrical power source and each aerosolisation means.

Preferably, the downstream pulse-width modulator 160 adjusts an alternating electrical duty cycle of each aerosolisation means as a function of the ratio determined, by alternating between two states referred to as “left” and “right”.

“Downstream pulse-width modulator” refers to the pulse-width modulator connected to the set of aerosolisation means and to the other pulse-width modulator. “Upstream pulse-width modulator” refers to the pulse-width modulator connected to the other pulse-width modulator and to the autonomous electrical power source.

“Left” state refers to the transmission of an electrical current to one of the aerosolisation means 120, and “right” state to the transmission of an electrical current to one of the aerosolisation means 125. “On” state refers to the transmission of an electrical current from an upstream point to a downstream point of an electrical circuit, and “off” state to the absence of transmission of an electrical current from said upstream point to said downstream point, in the manner of an electrical “on/off” switch.

The downstream pulse-width modulator operates at a lower frequency than the upstream pulse-width modulator.

It is noted that a duty cycle is defined, for a periodic signal, as the time during which a signal is at high state, i.e. an electron current passes, over a period, referred to here as “switching period”. It is also noted that the alternating electrical duty cycle of the aerosolisation means, 120 and 125, is not directly proportional to the quantity of liquid to be aerosolised for each liquid.

The downstream pulse-width modulator is configured to adjust the alternating duty cycle between the electrical power supply of one or other of the aerosolisation means, 120 and 125, without the set of aerosolisation means being supplied with electrical power at the same time. This means that the electrical energy supply signals of each aerosolisation means are synchronised over the same period: the switching period. And only the supply signal of one aerosolisation means, 120 or 125, is at high state at any one time.

In some embodiments, the supply signals of the aerosolisation means, 120 and 125, can be at the low state at the same time.

Therefore, over a switching period, the sum of the duty cycles of the supply signals of the aerosolisation means, 120 and 125, is less than or equal to one.

In some embodiments, the upstream pulse-width modulator 155 defines a duty cycle for supplying electrical energy to the set of aerosolisation means by alternating between two states, referred to as “on” and “off”.

The upstream pulse-width modulator 155 is connected to the autonomous electrical power source 130 and modulates the electric current from the autonomous electrical power source 130 to define an electrical power available to supply the set of aerosolisation means alternatively. The electrical power available depends on the average value of the electrical current obtained on output from the pulse-width modulator. The average value of the electrical current is directly proportional to the time during which the pulse-width modulator is in an on state over a switching period.

The downstream pulse-width modulator 160 is configured to switch the distribution of the current obtained on output from the first pulse-width modulator 155 between the possible states of the switching means 140, i.e. left or right, during the on state of the upstream pulse-width modulator 155. The switching depends on the adjusted duty cycles.

In these embodiments, each pulse-width modulator, 155 and 160, is synchronised with a clock signal that defines the switching period and connected to the autonomous electrical power source 130.

Over a switching period, the switching ratio between the supply signals of each aerosolisation means, 120 and 125, can be defined. The switching ratio is the ratio of the duration for which the first aerosolisation means 120 is supplied with power and the duration for which the second aerosolisation means 125 is supplied with power. The switching ratio is directly proportional to the duty cycles of the power signals of each aerosolisation means, 120 and 125.

The switching ratio depends on at least the duration of activation of the set of aerosolisation means, and therefore of inhalation, the duty cycle of the first aerosolisation means 120, the duty cycle of the second aerosolisation means 125, and the power supplying each aerosolisation means, 120 and 125. However the duty cycle is not linear with the quantity of aerosolised liquid to be evaporated from each tank.

In other embodiments, the switching ratio depends on:
an inhalation flow-rate;
an ambient temperature;
an ambient humidity;
the viscosity of each liquid;
at least one thermodynamic property of each liquid;
a ratio between propylene glycol (acronym “PG”) and vegetable glycerin (acronym “VG”);
the formulation of each liquid;
the value of the aerosolisation means in Ohms;
the surface area of each aerosolisation means, 120 and 125;
the material of each aerosolisation means, 120 and 125;
at least one thermodynamic property of each aerosolisation means, 120 and 125;
a type of each aerosolisation means, 120 and 125, for example, a wire, a mesh, a ceramic element;
a shape and dimensions of each aerosolisation means, 120 and 125;
a position of an element impregnated with liquid in relation to at least one aerosolisation means, 120 and 125;
a thermal capacity of each aerosolisation means, 120 and 125; and/or
the design of the air channel, i.e. the way in which the incoming air arrives on the heating element and travels a path up to the inhalation means 115.

The inventors have calculated the switching ratio, with regard to the use of heating resistors as aerosolisation means, by performing tests on standardised series. The experiments performed are described below.

A standardised series is defined by twenty artificial inhalations, each with a duration of three seconds and a flow-rate of 55 mL, spaced by a thirty-second wait between each artificial inhalation. The set of thirty cycles is repeated three times, i.e. forming a triplet.

The purpose of the first part of the protocol is to determine the maximum power supplied to each aerosolisation means to avoid a dry hit. Typically, in an electronic cigarette, an aerosolisation means surrounds a cotton wick impregnated with liquid. A dry hit produces a burnt taste due to the overheating of the aerosolisation means when too little liquid is available to supply the cotton wick in contact with the aerosolisation means.

The maximum power determined is valid for the thermodynamic system studied during the test. A change to the thermodynamic properties of the device can lead to a new calculation of the maximum power.

To begin with, a single tank is therefore used:

1) The tank used is weighed after being filled. A median voltage of 3.6V is first used on a standardised series. For each inhalation it is noted whether there was a dry hit or not. At the end of the series, the tank is weighed to know the quantity of evaporated liquid.

2) If no dry hit was detected, step 1) is repeated with the voltage increased by 0.1 Volt (V) until a dry hit is obtained. The voltage before the iteration producing a dry hit is therefore the maximum voltage.

3) The maximum voltage has therefore been determined for a 3-second inhalation time. So it is now necessary to verify that no dry hit occurs with longer inhalation times. Step 1) is therefore repeated using the voltage found previously, but with an inhalation time of 5 seconds.

4) If no dry hit has been detected, step 1) is repeated using the maximum voltage, but with an inhalation time, otherwise referred to as “puff” in a way known to the person skilled in the art, of 7 seconds.

5) If no dry hit has been detected, the maximum voltage on a tank has therefore been determined with the quantity of evaporated liquid for a duration of 3, 5 and 7 seconds.

In a second step, two tanks are used at the same time with a switching ratio of 50%:

1) The two tanks are weighed. A standardised series with a puff duration of 3 seconds is performed with the maximum voltage. The two tanks are weighed to know the quantity of evaporated liquid.

2) While the quantity of evaporated liquid is not equal to the quantity of evaporated liquid obtained with a single tank, step 1) is repeated with the voltage increased.

3) Once this voltage has been found for an inhalation time of 3 seconds, this test is repeated to find the voltage for a duration of 5 seconds and 7 seconds.

The following table can be established:

| | | Maximum voltage for a |
|---|---|---|
| | Maximum voltage for a | switching ratio of 50% |
| | single aerosolisation | between two aerosolisation |
| | means | means |
| Standardised | X1max | X2max |
| series | | |
| (3 secs) | | |
| Standardised | Y1max | Y2max |
| series | | |
| (5 secs) | | |
| Standardised | Z1max | Z2max |
| series | | |
| (7 secs) | | |

Note that the voltage X2max with a duty cycle of 50% makes it possible to obtain the same quantity of liquid evaporated as the voltage X1max with a duty cycle of 100%.

Next, the impact of the switching ratio on the ratio of evaporated liquid is determined. The purpose of the second part of the protocol is to determine the ratios of evaporated liquid as a function of the switching ratio for each set duration and voltage pair.

A standardised series is performed with the duty cycle being varied until a ratio of evaporated liquid of 97.5% for one liquid and 2.5% of the other liquid is achieved. The following table is obtained:

| | Concentration | Left | Right | | | |
|---|---|---|---|---|---|---|
| | of | duty | duty | Liquid | Left | Right |
| Duration | Nicotine | cycle | cycle | quantity | ratio | ratio |
| 3 sec | 10 | 50 | 50 | 0.57 | 49.93% | 50.07% |
| | 9.5 | 52.5 | 47.5 | 0.54 | 59.67% | 40.33% |
| | 9 | 55 | 45 | 0.54 | 66.46% | 33.54% |
| | 8.5 | 57.5 | 42.5 | 0.54 | 70.98% | 29.02% |
| | 8 | 60 | 40 | 0.55 | 74.00% | 26.00% |
| | 7.5 | 62.5 | 37.5 | | | |
| | 7 | 65 | 35 | | | |
| | 6.5 | 67.5 | 32.5 | | | |
| | 6 | 70 | 30 | | | |
| | 5.5 | 72.5 | 27.5 | | | |
| | 5 | 75 | 25 | | | |
| | 0 | 100 | 0 | | | |

This table shows, for example, that a switching ratio of 60% for the supply duration of the first aerosolisation means and 40% for the supply duration of the second aerosolisation means over a switching period causes an evaporation of 74% for the first liquid and 26% for the second liquid.

The evaporation of liquid is not linear with the switching ratio, and the inequality of the liquid ratio increases exponentially as the inequality of the switching ratio increases.

The experiment is repeated for all the duration and maximum voltage pairs, and for any new thermodynamic system.

Lastly, the data are compiled and a mathematical formula is determined to obtain a switching ratio as a function of a ratio of liquid to be evaporated. At this stage we therefore have a table comprising a large number of entries which indicates the ratio of evaporated liquid as a function of each parameter: i.e., at least, the voltage applied to each aerosolisation means and the switching ratio.

Dependent and regressive variables are defined according to the model of a linear regression sequence. A mathematical formula is then obtained as a function of different parameters and a ratio for the pertinence of this formula, e.g. an accuracy or an error rate. If the pertinence ratio data are acceptable, we can therefore proceed to the step of validating this formula.

In this step, the formula is validated theoretically for all the parameters and then by comparing the results found against the results of experiments carried out previously. If the results obtained are similar to the actual results, then this means that the formula is a good match and the formula is implemented by the switching means 140.

A new series of tests is performed with the quantity of evaporated liquid measured. This new series makes it possible to validate that the wished-for ratio of evaporated liquid has been obtained, whatever the inhalation duration and voltage applied to the aerosolisation means. If the theoretical results obtained have differences from the actual results, the mathematical formula is re-assessed.

In some embodiments, the device 10 comprises a means 165 for calculating an inhalation time and a means 170 for adjusting the switching as a function of the inhalation time calculated.

In some embodiments, the calculated inhalation time is learned from user data. For example, an average inhalation time can be calculated using the last 500 inhalations by the user. The calculation means 165 then injects the average time calculated into the mathematical formula obtained to calculate the switching ratio.

These embodiments can generate an error when the inhalation time is not equal to the average time.

In some embodiments, the inhalation time is calculated using a current inhalation and the means 170 for adjusting the switching adjusts the switching dynamically. In these embodiments, the inhalation time is measured and the switching ratio is adjusted every 0.1 seconds, for example.

In effect, for a set voltage, or power and switching ratio, the ratio of evaporated liquid changes as a function of the inhalation time. The switching ratio is readjusted every 0.1 seconds as a function of the previously obtained mathematical formula.

FIG. 6 shows a second embodiment of a device that is the subject of the present invention. In the embodiment shown in FIG. 6, the switching means 640 comprises a single pulse-width modulator 650 between the autonomous electrical power source and each aerosolisation means. The rest of the device operates in the same way as described above.

The switching means 640 manages the electrical power supply to each aerosolisation means. The switching means 640 switches the electrical power supply between three states, a first referred to as “left” configured to supply a first aerosolisation means 120 with electrical power, a second referred to as “right” configured to supply a second aerosolisation means 125 with electrical power, and a third state referred to as “off” in which no aerosolisation means is not supplied with electrical power.

The pulse-width modulator (650) has three supply states, “off”, “left” and “right”, and the pulse-width modulator adjusts:
a duty cycle for supplying electrical energy by alternating between the sum of the duration in the left and right states, and the duration in the off state; then
an alternating duty cycle for supplying each aerosolisation means with electrical power as a function of the ratio determined, by alternating between the two supply states, referred to left” and “right”. The duty cycle for supplying electrical energy to the aerosolisation means by alternating between an off state and an on state, is calculated according to the power to be delivered based on the voltage of the battery. Once the on or off duration has been calculated, an alternating duty cycle for supplying each aerosolisation means with electrical power is calculated as a function of the ratio determined and applied over the time of the on period.

Therefore, over a switching period, the sum of the duty cycles of the supply signals of the aerosolisation means, 120 and 125, is less than or equal to one.

The pulse-width modulator 650 is connected to the autonomous electrical power source 130 and modulates the electric current from the autonomous electrical power source 130 to define an electrical power available to supply the set of aerosolisation means alternately. The electrical power available depends on the average value of the electrical current obtained on output from the pulse-width modulator. The average value of the electrical current is directly proportional to the time during which the pulse-width modulator is in an on state over a switching period. An “on” state corresponds to a “left” or “right” state.

The pulse-width modulator 650 is also configured to switch the distribution of the current obtained between the left or right states during the on state of the upstream pulse-width modulator 650. The switching depends on the adjusted duty cycles.

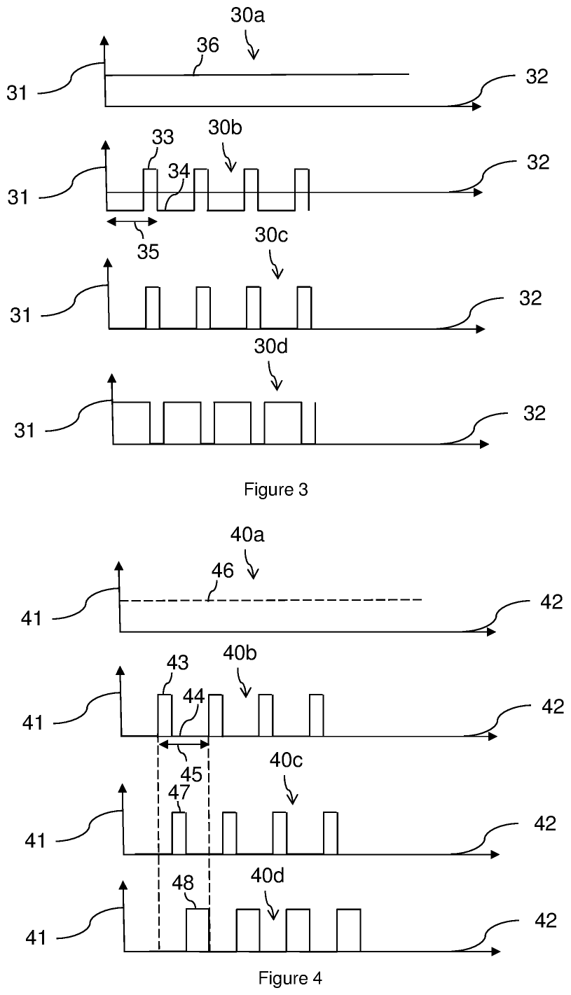

With regard to the device 10, FIG. 3 shows electrical supply diagrams for the switching means 140 and each aerosolisation means, 120 and 125, as a function of time 32. FIG. 3 shows the electrical power supply for each aerosolisation means, 120 and 125, as a function of time 32 when two pulse-width modulators, 155 and 160, are installed in series.

Diagram 30a shows the average voltage from the pulse-width modulator. Diagram 30b shows the electrical power supply of the downstream pulse-width modulator 160. Diagram 30c shows the electrical power supply of the first aerosolisation means 120 and diagram 30d shows the electrical power supply of the second aerosolisation means 125. Diagrams 30a, 30b, 30c and 30d show four switching periods 35.

Diagram 30a shows an average voltage 36 with a predefined value. The predefined value depends on the time during which a high state is defined for the pulse-width modulator in relation to the period.

Diagram 30b shows the voltage from the second pulse-width modulator 160 is at a left state 33 and a right state 34 for a predefined duty cycle, and preferably the number of left and right states are equal and opposite in sign.

For the same duty cycle, when the voltage has a positive sign in FIG. 30b, the aerosolisation means 120 is supplied with electrical power. And when the voltage has a negative sign for the switching means 140, the aerosolisation means 125 is supplied with electrical power.

With regard to the device 10, FIG. 4 shows electrical supply diagrams for the switching means 640 and each aerosolisation means, 120 and 125, as a function of time 32.

Diagram 40a shows the average voltage from the autonomous electrical power source 130. Diagram 40b shows the electrical power supply of the first aerosolisation means 120 when the pulse-width modulator 650 is in the “left” state. Diagram 40c shows the electrical power supply of the second aerosolisation means 125 when the pulse-width modulator 650 is in the “right” state. And diagram 40d shows the electrical power supply of the second aerosolisation means 125 when the pulse-width modulator 650 is in the “off” state. Diagrams 40a, 40b and 40c show four switching periods 35.

Diagram 40a shows an average voltage 46 with a predefined value available to be distributed. It is therefore an average value dependent on the time over a period 45 during which the pulse-width modulator is in the state “on”, i.e. “left” or “right”.

For the same duty cycle for each aerosolisation means, 120 and 125, when the voltage has a positive sign in FIG. 40b, the aerosolisation means 120 is supplied with electrical power. Similarly, when the voltage has a positive sign in FIG. 40c, the aerosolisation means 125 is supplied with electrical power. It can be seen that when the voltage in FIG. 40b reaches a falling edge, the voltage in diagram 40c has a rising edge to avoid the two aerosolisation means being supplied at the same time.

The same duty cycle for each aerosolisation means, 120 and 125, means the same average voltage value can be applied to each aerosolisation means, 120 and 125.

In some embodiments, the device 10 comprises a means for capturing an item of data representative of a temperature in at least one tank, 105 and 110. This capture means 155 is, for example, an electronic thermometer. In some embodiments, the control means 11 controls the heating of the aerosolisation means, 120 and 125, associated with each said tank, 105 and 110, according to the temperature captured.

In some variants, the device 10 comprises a means for capturing the inhalation flow-rate of a user. This means for capturing the flow-rate is, for example, an electronic circuit connected to a propeller positioned in the duct. On the basis of a captured rotation of the propeller and of a predefined value representative of the surface area of the cross-section of the duct at the location of the propeller, the means for capturing the flow-rate calculates the inhalation flow-rate.

In some variants, the device 10 comprises a means for emitting the user's consumption information to a remote memory. This emission means is, for example, an antenna configured to emit a wireless signal using, for example, standard IEEE 802.11, known as “Wi-Fi”. The consumption information memorised in this way makes it possible, for example, to establish statistics transmitted to a communicating portable terminal of the user.

In some variants, the device 10 comprises a screen for displaying information representative of:
a charge level of the battery;
a filling level of one or of each tank;
a mode of consumption, manual or automatic, of the active substance; and/or
a heating ratio between the two aerosolisation means;
a value of the set of aerosolisation means detected in Ohms;
wear of the set of aerosolisation means as a percentage;
a real-time temperature of the set of aerosolisation means;
a total power or total voltage at the terminals of each aerosolisation means;
a concentration of active substance to be aerosolised;
a volume of aerosolised liquid to be produced for each tank, for example a ratio;
a value of the heating power ratio between the set of aerosolisation means; and/or
various messages in the form of text.

In some variants, the device 10 comprises a means for emitting a light signal. This means for emitting a light signal is, for example, a light-emitting diode configured to emit light when a detected inhalation frequency of the user is higher than a predefined limit value.

In some variants, the control means 11 is deactivated during a predefined limit time when a predefined limit quantity or concentration of active substance has been aerosolised during a predefined limit time.

In some variants, at least one of the tanks, 105 and 110, comprises a medicine configured to be taken orally or by inhaler. This medicine is, for example, in the form of large molecules broken up by a means for emitting ultrasounds.

In some variants, the determination means 130 determines a quantity of active substance to be inhaled as a function of an item of information about an event, declared by the user, related to an item of timestamp data. When the determination of a quantity of active substance occurred during the memorised event, the determined quantity of active substance is increased.

In some variants, the end piece for inhalation 115 is connected to a geolocation means and an item of data representative of a location is associated in a memory with each item of data for an inhalation.

In some variants, at least one emission means emitting a signal using Bluetooth technology utilises Bluetooth Low Energy technology.

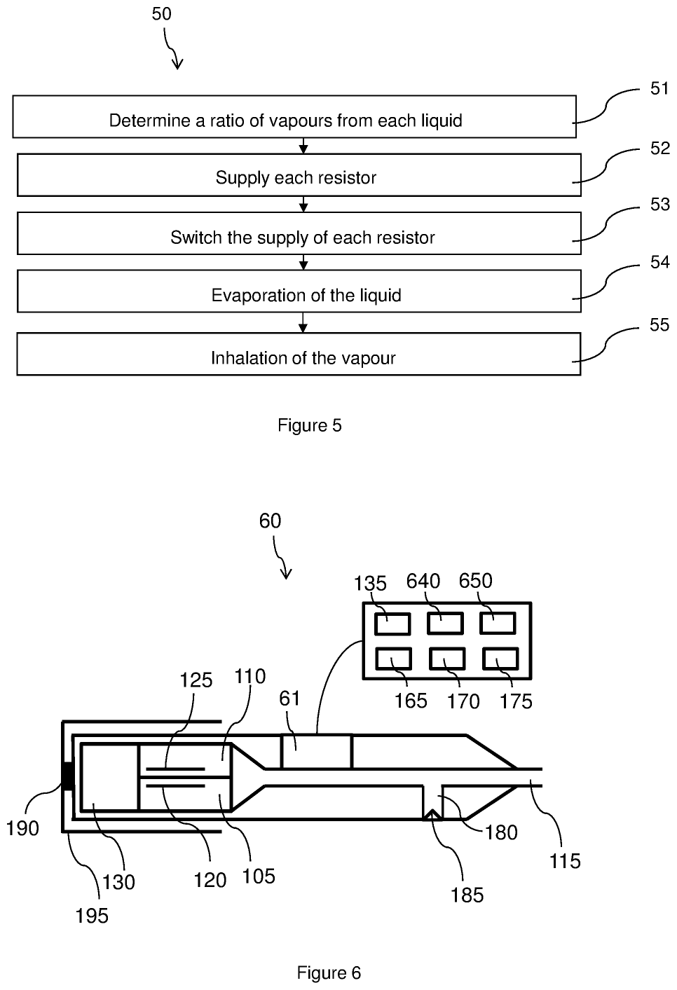

FIG. 5 shows a particular embodiment of a method 50 of adjusting the quantity of two aerosolised liquids to be inhaled simultaneously by a user, each liquid being contained in a tank associated with one aerosolisation means, which comprises:
a step 51 of determining a ratio of liquids to be aerosolised for each liquid;
a switching step 52 for alternately supplying each aerosolisation means with electrical power from the single autonomous power source, by pulse-width modulation between the autonomous electrical power source and each aerosolisation means by the use of one pulse-width modulator or two pulse-width modulators installed in series;
a step 53 of evaporating the liquid contained in each tank when this liquid undergoes a determined physical interaction; and
a step 54 of inhaling, by the user, aerosolised liquid coming from each tank.

The means of the devices 10 and are configured to utilise the steps of the method 50 and their embodiments as described above, and the method 50 and its various embodiments have steps corresponding to the means of devices 10 and 60.

1. Device for adjusting the quantity of two liquids that are aerosolised in order to be inhaled simultaneously by a user, characterised in that said device comprises:
two tanks, a first tank comprising a first liquid and a second tank comprising a second liquid having at least one different property, each liquid being configured to be aerosolised when this liquid undergoes a determined physical interaction;
an end piece for inhalation, by the user, of the aerosolised liquid coming from each tank;
two aerosolisation means for aerosolising the first and second liquid respectively, each tank being associated with one aerosolisation means;
a single autonomous source of electrical power for supplying electrical power to each aerosolisation means;
a means for determining a ratio of liquids to be aerosolised for each liquid; and
a switching means for alternately supplying each aerosolisation means with electrical power from the single autonomous power source, as a function of the determined ratio, the switching means comprising two pulse-width modulators installed in series between the autonomous electrical power source and each aerosolisation means.

2. Device according to claim 1, wherein the upstream pulse-width modulator defines a duty cycle for supplying electrical energy to the aerosolisation means by alternating between two states, referred to as “on” and “off”.

3. Device according to claim 1, wherein the downstream pulse-width modulator adjusts an alternating electrical duty cycle of each aerosolisation means as a function of the ratio determined, by alternating between two states referred to as “left” and “right”.

4. Device according to claim 1, which also comprises a means for calculating an inhalation time and a means for adjusting the switching as a function of the inhalation time calculated.

5. Device according to claim 4, wherein the calculated inhalation time is learned from user data collected.

6. Device according to claim 5, wherein the inhalation time is calculated using a current inhalation and the means for adjusting the switching adjusts the switching dynamically.

7. Device according to claim 1, which also comprises a means for choosing a ratio between a quantity of air or a quantity of aerosolised liquid to be inhaled, and in which the end piece for inhalation comprises an air inlet and a means for closing the air inlet as a function of the ratio chosen.

8. Device for adjusting the quantity of two liquids that are aerosolised in order to be inhaled simultaneously by a user, characterised in that said device comprises:
two tanks, a first tank comprising a first liquid and a second tank comprising a second liquid having at least one different property, each liquid being configured to be aerosolised when this liquid undergoes a determined physical interaction;
an end piece for inhalation, by the user, of the aerosolised liquid coming from each tank;
two aerosolisation means for aerosolising the first and second liquid respectively, each tank being associated with one aerosolisation means;
a single autonomous source of electrical power for supplying electrical power to each aerosolisation means;
a means for determining a ratio of liquids to be aerosolised for each liquid; and
a switching means for alternately supplying each aerosolisation means with electrical power from the single autonomous power source, as a function of the determined ratio, the switching means comprising a single pulse-width modulator between the autonomous electrical power source and each aerosolisation means.

9. Device according to claim 8, wherein the pulse-width modulator has three supply states, “off”, “left” and “right”, and the pulse-width modulator adjusts:
a duty cycle for supplying electrical energy by alternating between the sum of the duration in the left and right states, and the duration in the off state; then
an alternating duty cycle for supplying each aerosolisation means with electrical power as a function of the ratio determined, by alternating between the two supply states, left and right.

10. Device according to claim 8, which also comprises a means for calculating an inhalation time and a means for adjusting the switching as a function of the inhalation time calculated.

11. Device according to claim 11, wherein the calculated inhalation time is learned from user data collected.

12. Device according to claim 12, wherein the inhalation time is calculated using a current inhalation and the means for adjusting the switching adjusts the switching dynamically.

13. Device according to claim 9, which also comprises a means for choosing a ratio between a quantity of air or a quantity of aerosolised liquid to be inhaled, and in which the end piece for inhalation comprises an air inlet and a means for closing the air inlet as a function of the ratio chosen.

14. Method for adjusting the quantity of two liquids that are aerosolised in order to be inhaled simultaneously by a user, characterised in that said device comprises:
a step of determining a ratio of liquids to be aerosolised for each liquid;
a switching step for alternately supplying each aerosolisation means with electrical power from the single autonomous power source, by pulse-width modulation between the autonomous electrical power source and each aerosolisation means by the use of one pulse-width modulator or two pulse-width modulators installed in series;
a step of evaporating the liquid contained in each tank when this liquid undergoes a determined physical interaction; and
a step of the user inhaling aerosolised liquid coming from each tank.
